SEQUENCE LISTING (20 sequences)

SEQ ID NO: 1 - protein, 15 aa, synthetic construct
  REGION 1..15: note = Synthetic Peptide
MVRIKPASANKPSDD

SEQ ID NO: 2 - protein, 15 aa, synthetic construct
  REGION 1..15: note = Synthetic Peptide
MVRIKPGTANKPSDD

SEQ ID NO: 3 - protein, 15 aa, synthetic construct
  REGION 1..15: note = Synthetic Peptide
MVRIKPGTANKPTDD

SEQ ID NO: 4 - protein, 15 aa, synthetic construct
  REGION 1..15: note = Synthetic Peptide
MVRIKPGSGNKPSDD

SEQ ID NO: 5 - protein, 15 aa, synthetic construct
  REGION 1..15: note = Synthetic Peptide
MVRIKPATANKPSDD

SEQ ID NO: 6 - protein, 15 aa, synthetic construct
  REGION 1..15: note = Synthetic Peptide
MVRIKPGTGNKPSDD

SEQ ID NO: 7 - protein, 15 aa, synthetic construct
  REGION 1..15: note = Synthetic Peptide
MVRIKPGTGNKPTDD

SEQ ID NO: 8 - protein, 15 aa, synthetic construct
  REGION 1..15: note = Synthetic Peptide
MVRIKPATANKPTDD

SEQ ID NO: 9 - protein, 15 aa, synthetic construct
  REGION 1..15: note = Synthetic Peptide
MVRIKPATGNKPTDD

SEQ ID NO: 10 - protein, 15 aa, synthetic construct
  REGION 1..15: note = Synthetic Peptide
MVRIKPATGNKPSDD

SEQ ID NO: 11 - protein, 14 aa, synthetic construct
  REGION 1..14: note = Synthetic Peptide
VRLKPASANKPSED

SEQ ID NO: 12 - protein, 14 aa, synthetic construct
  REGION 1..14: note = Synthetic Peptide
VRLKPASGNKPSED

SEQ ID NO: 13 - protein, 14 aa, synthetic construct
  REGION 1..14: note = Synthetic Peptide
VRLKPGSGNKPSED

SEQ ID NO: 14 - protein, 13 aa, synthetic construct
  REGION 1..13: note = Synthetic Peptide
VRLKPATGNKPTE

SEQ ID NO: 15 - protein, 14 aa, synthetic construct
  REGION 1..14: note = Synthetic Peptide
VRLKPGTANKPTED

SEQ ID NO: 16 - protein, 14 aa, synthetic construct
  REGION 1..14: note = Synthetic Peptide
VRLKPATANKPTED

SEQ ID NO: 17 - protein, 14 aa, synthetic construct
  REGION 1..14: note = Synthetic Peptide
VRLKPGTGNKPTED

SEQ ID NO: 18 - protein, 14 aa, synthetic construct
  REGION 1..14: note = Synthetic Peptide
VRLKPGTANKPSED

SEQ ID NO: 19 - protein, 14 aa, synthetic construct
  REGION 1..14: note = Synthetic Peptide
VRIKPGTANKPSED

SEQ ID NO: 20 - protein, 15 aa, synthetic construct
  REGION 1..15: note = Synthetic Peptide
  VARIANT 1: note = X can be M or no amino acid
  VARIANT 4: note = X can be I or L
  VARIANT 7: note = X can be G or A
  VARIANT 8: note = X can be S or T
  VARIANT 9: note = X can be A or G
  VARIANT 13: note = X can be S or T
  VARIANT 14: note = X can be D or E
XVRXKPXXXNKPXXD

(19) United States
(12) Patent Application Publication
(10) Pub. No.: US 2026/0102458 A1
(43) Pub. Date: Apr. 16, 2026

(54) METHODS OF TREATING LONG TERM CORONAVIRUS INFECTION

(71) Applicant: UNIVERSITY OF MIAMI, Miami, FL (US)

(72) Inventors: Michael Paidas, Miami, FL (US); Arumugam Jayakumar, Miami, FL (US)

(21) Appl. No.: 19/213,302

(22) Filed: May 20, 2025

Related U.S. Application Data
(60) Provisional application No. 63/650,100, filed on May 21, 2024.

(51) Int. Cl. A61K 38/10; A61P 31/14

(52) U.S. Cl. CPC A61K 38/10; A61P 31/14

(57) ABSTRACT

Described herein is a method of treating or preventing a long term coronavirus infection in a subject in need thereof comprising a therapeutically effective amount of a peptide, wherein the formulation comprises peptides having an amino acid sequence comprising: (M)nVR(I/L)KP(G/A)(S/T)(A/G)NKP(S/T)(D/E)D, (SEQ ID NO: 20), where n=0 or 1.

### INCORPORATION BY REFERENCE OF MATERIALS SUBMITTED ELECTRONICALLY

This application contains, as a separate part of the disclosure, a Sequence Listing in computer readable form (Filename: 70310_SubSeqlisting.txt; Size: 28,571 bytes; Created: Jul. 28, 2025), which is incorporated by reference in its entirety.

### BACKGROUND

The COVID-19 pandemic has been one of the most impactful events in our lifetime, caused by severe acute respiratory syndrome coronavirus-2 (SARS-CoV-2). Multiple SARS-CoV-2 variants were reported globally, and a wide range of symptoms existed. Individuals who contract COVID-19 continue to suffer for a long time, known as long COVID or post-acute sequelae of COVID-19 (PASC). While COVID-19 vaccines were widely deployed, both unvaccinated and vaccinated individuals experienced long-term complications. To date, there are no treatments to eradicate long COVID.

### BRIEF DESCRIPTION OF THE FIGURES

FIG. 1. SPIKENET Structure and Binding Affinity to the ACE2 Binding Domain of the SARS-CoV-2 Spike Glycoprotein. Structure of SPIKENET, a 15 amino acid synthetic peptide targeted to the ACE2 binding domain of the SARS-CoV-2 spike glycoprotein (top image). The computational protein docking approach shows particular SPIKENET binding affinity to the ACE2 and CEACAM1 binding domains of the SARS-CoV-2 and MHV-1 spike glycoproteins (S1), respectively (bottom images) [14].

FIG. 2. Spectroscopic analysis shows highly specific SPIKENET (SPK) binding affinity to the ACE2 binding domain of the SARS-CoV-2 spike glycoprotein (S1)-Ab, absorbance; WI, wavelength.

FIG. 3. The LIGPLOT diagram of the protein-peptide interaction between the RBD and SPIKENET. The pink SPIKENET peptide residues are shown on the top, and the yellow amino acid residues of the RBD from SARS-CoV-2-two are shown at the bottom. The SPIKENET peptide consists of 15 amino acid residues, 14 of which are shown in non-bonded interactions with the RBD of SARS-CoV-2, proving that SPIKENET has a significant binding affinity with the spike glycoprotein-1. These findings strongly suggest that SPIKENET is a potent competitive inhibitor of S1.

FIG. 4. The protein docking approach shows SPIKENET binding affinity to the CEACAM1a binding domain of the MHV-1 spike protein.

FIG. 5. SPIKENET binding affinity to the CEACAM1a (CCM) binding domain of the MHV-1-NTD by molecular dynamic studies. The RMSD of both complexes with their respective native proteins are shown in (A, B). The structures showed complete equilibration in the system when comparing the RMSD of both NTD and NTD+SPK (A) after 50 ns dynamic simulation. The SPK peptide was well stabilized and had a high affinity at the CCM binding location of NTD. However, the RMSD analysis of CCM and CCM+SPK structures after the 50 ns dynamic simulation exhibited more flexibility at the NTD binding site of CCM than native CCM (B), suggesting the SPK peptide detachment and displacement over the CCM.

FIG. 6. MHV-1-inoculated mice lost 20-40% of body weight over days 3-7. Treatment of MHV-1-inoculated mice with SPIKENET (3 doses, on days 2, 4, and 6 post MHV-1 injection with 5 mg/kg) reversed such loss. These findings correlated well with the animal survival post-SPIKENET treatment in SARS-CoV-2-infected K18-hACE2 transgenic mice (n=10).

FIG. 7. SPIKENET treatment reduces the death rate in MHV-1-infected mice. Female A/J mice were inoculated with MHV-1 intranasally. SPIKENET (5 mg/kg b.wt.) was injected subcutaneously when mice showed sickness (i.e., 2 days after MHV-1 exposure). SPIKENET was injected 2 times every alternate day. SPIKENET diminished the animal's death.

FIG. 8. Elevated edema was observed in MHV-1 infected brain, lung, liver, kidney, and heart, as compared to control, which was similar to patients with COVID-19. Treatment of MHV-1-infected mice with SPK (5 mg/kg; 3 injections from 2 to 6 days) showed edema levels comparable to control levels on day 7.

FIGS. 9A and 9B. Oxidative stress post-MHV-1 infection in mice. (FIG. 9A) Representative immunofluorescence images from four individual animals show increased levels of 4-hydroxynonenol (4-HNE) and malondialdehyde (MDA) in the lung, liver, kidney, brain, and heart. Treatment of MHV-1 inoculated mice with SPK (5 mg/kg) prevented such an increase. (FIGS. 9B, 9C) Quantitation of 4-HNE and MDA levels with and without SPK post-MHV-1 infection. ANOVA, n=4. *p<0.05 versus control; †p<0.05 verses MHV-1 infected mice. Scale bar=25 μm. Error bars represent mean±SEM.

FIGS. 10A-10E. Effect of SPIKENET (SPK) on hydrogen peroxide (H2O2)-induced oxidative stress (protein carbonyl formation) (12 h) (FIG. 10A), and LPS-induced LDH release (36 h) (FIG. 10B) in primary cultures of rat brain microglia, as well as H2O2-induced increase in cell volume (24 h) in primary cultures of rat brain astrocytes (FIG. 10C). SPK significantly diminished these effects in glial cells (30 min post-treatment). Exposure of primary microglia to LPS (24 h) showed an increase in DCF fluorescence (FIG. 10D), as well as an increase in IL-6 level (FIG. 10E) in cell culture medium, and such increase was diminished and blocked by treatment of cells (30 min post-treatment) with SPIKENET. C, control; LPS, lipopolysaccharide. *p<0.05 vs. control. †p<0.05 vs. LPS. C, control; AIU, arbitrary intensity units; LPS, lipopolysaccharide. Error bars represent mean±SEM.

FIG. 11. Altered AQP levels were identified in various organs post-MHV-1 inoculation. While increased AQP1 has been identified in multiple organs, AQP1 levels were decreased in lungs post-MHV-1. Further, treatment of MHV-1 inoculated mice with SPK (5 mg/kg) reversed these changes. Scale bar=25 μm.

FIG. 12. SPIKENET (SPK) diminishes MHV-1-induced pathological changes in the brain, lungs, heart, liver, kidney, and skin during long-term infection. Representative histological images of hematoxylin and eosin (H&E) stained brain, lung, heart, liver, kidney, and skin tissue sections of a standard mouse (Frames A, D, G, J, M, P) and an infected mouse at 12 months (Frames B, E, H, K, N, Q). MHV-1 inoculated mice treated with 5 mg/kg SPIKENET (SPK) eased all of these changes (Frames C, F, I, L, O, R) (H&E original magnification is 400× for Frames A-O, and 66× for Frames P-R).

FIGS. 13A-13C. Altered mRNA expressions 12 months post-MHV-1 infection (chronic). (FIG. 13A) EGFR mRNA level does not significantly change in disease and treatment with SPK. (FIG. 13B) TGF-β1 mRNA level is significantly increased in the infected group, while its mRNA level is decreased substantially following SPK treatment. (FIG. 13C) FGF-23 mRNA level is significantly increased in the infected group, and therapy with SPK leads to substantial elevation. Values are the mean SD of three independent experiments. *=p<0.05 is statistically significant. ns=nonsignificant.

FIG. 14. The MHV-1 coronavirus-mediated signaling systems in various organs result in pathological and functional consequences. SPK ameliorated or prevented such defects in these organs.

FIG. 15. MHV-1 coronavirus instigated signaling pathways across diverse organs, leading to pathological and functional repercussions. SPK mitigated or averted such abnormalities within these organs.

### DETAILED DESCRIPTION

We recently conceived a new approach to treat COVID in which a 15 amino acid synthetic peptide (SPIKENET, SPK) is targeted to the ACE2 receptor binding domain of the SARS-CoV-2, which prevents the virus from attaching to the host. We also found that SPK precludes the binding of spike glycoproteins with the receptor carcinoembryonic antigen-related cell adhesion molecule 1 (CEACAM1) of a coronavirus, the murine hepatitis virus-1 (MHV-1), and with all SARS-CoV-2 variants. Further, SPK reversed the development of severe inflammation, oxidative stress, tissue edema, and animal death post-MHV-1 infection in mice. SPK also protects against multiple organ damage in acute and long-term post-MHV-1 infection. Our findings collectively suggest a potential therapeutic benefit of SPK for treating COVID-19.

### Definitions

The term “severe acute respiratory syndrome virus” (SARS-CoV-2) refers to the virus responsible for the Coronavirus disease 2019 (COVID-19) global pandemic. SARS-CoV-2 infection is also referred to as “COVID-19” and SARS-CoV-2 is “the virus responsible for COVID-19”. In addition, the current disclosure also encompasses human coronavirus species strains in addition to SARS-CoV-2. These include SARS-CoV-2 variants, additional SARS-CoV strains and the Middle East respiratory syndrome coronavirus (MERS-CoV).

The term “Spike glycoprotein” or “S protein” (SGP) or (S1) refers to the spike proteins that are anchored in the lipid bilayer of the viral envelope. The lipid bilayer also contains envelope (E) and membrane (M) proteins. The S protein are important in allowing interaction of the coronavirus with the host cell. The peptide of the current disclosure targets the S protein but may also be effective against host cells that interact with the virus via the M and E proteins.

As used herein the term “aquaporin” (AQP) or “water channel” means channel proteins that form pores in the membrane of biological cells, thereby facilitating transport between cells. Aquaporins, help facilitate the movement of water, a polar molecule, in addition to simple diffusion of water. Disruption of normal AQP structure and/or function may be associated with a wide range of diseases and conditions. Disruption in AQP or water channels often manifests as water accumulation and includes, but is not limited to venous insufficiency, heart failure, renal disease, low protein levels, liver problems, deep vein thrombosis, infections, angioedema, certain medications, lymphedema, menstruation, pregnancy, neuromyelitis optica, other viral infections, burn etc.

The term “patient” or “subject” as used herein includes human and animal subjects.

The term “disorder” is any condition that would benefit from treatment using the peptide of the disclosures. “Disorder” and “condition” are used interchangeably herein and include chronic and acute disorders or diseases, including those pathological conditions that predispose a patient to the disorder in question.

The terms “treatment” or “treat” refers to both therapeutic treatment and prophylactic or preventative measures. Those in need of treatment include those having the disorder as well as those prone to have the disorder or those in which the disorder is to be prevented.

The terms “pharmaceutical composition” or “therapeutic composition” or “pharmaceutical formulation” refers to a compound or composition capable of inducing a desired therapeutic effect when properly administered to a patient.

As used herein, the term “pharmaceutically acceptable carrier” or “physiologically acceptable carrier” refers to one or more formulation materials suitable for accomplishing or enhancing the delivery of the peptide. Such therapeutic or pharmaceutical compositions can comprise a therapeutically effective amount of a peptide, in admixture with a pharmaceutically or physiologically acceptable formulation agent selected for suitability with the mode of administration

Acceptable formulation materials preferably are nontoxic to recipients at the dosages and concentrations employed. The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropylbeta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides preferably sodium or potassium chloride- or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed, Mack Publishing Company 1990), and subsequent editions of the same, incorporated herein by reference for any purpose). The optimal pharmaceutical composition will be determined by one of skill in the art, accounting for the intended route of administration, delivery, format, and desired dosage. Aqueous or non-aqueous pharmaceutical compositions and/or vehicles can be used in the current disclosure. Examples of suitable injection vehicles or carriers include but is not limited to water physiological saline solutions such as phosphate buffered saline, and/or serum. Carriers can be supplemented with supplemented as needed with, for examples, vitamins, minerals, or electrolytes. Buffers, including TRIS buffers can be used. Buffers suitable for use have a pH of about 7.0-8.5 (TRIS buffers). The pharmaceutical compositions of the current disclosure can be mixed and prepared for short- or long-term storage using techniques known by those of skill in the art or prepared and used immediately.

As used herein the term “effective amount” and “therapeutically effective amount” when used in reference to a pharmaceutical composition comprising the peptide refers to an amount or dosage sufficient to produce a desired therapeutic result. The effective amount may vary depending on a variety of factors and conditions related to the patient being treated and the severity of the disorder. For example, if the peptide is to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

### Compositions and Formulations

The disclosure relates to reagents that are peptides each having a specific amino acid sequence. The disclosure also provides compositions, reagents, pharmaceutical formulations comprising such reagents, and methods for preventing and treating viral infections in humans including coronavirus infections, and in particular COVID-19 infections in humans. Further, the disclosure also provides pharmaceutical formulations of such peptide, alone or in combination with suitable excipients and other pharmaceutical formulating agents.

In some embodiments, the peptide comprises the amino acid sequence MVRIKPASANKPSDD (SEQ ID NO. 1) and is referred to herein as SPIKENET (SPK).

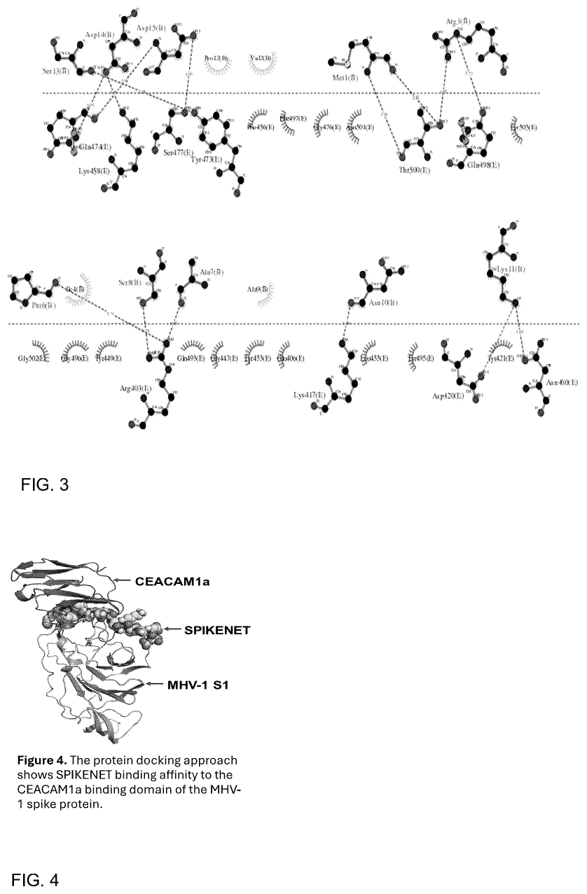

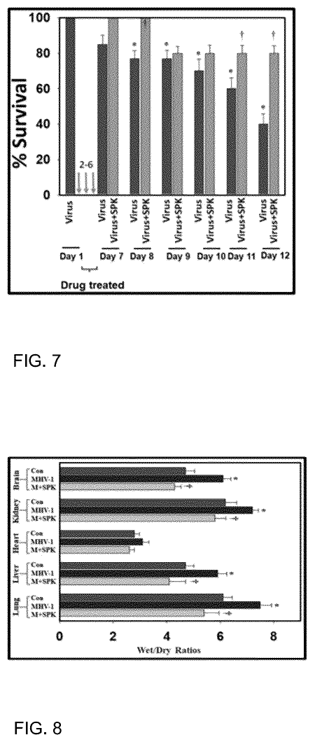

Cluspro computational protein docking program illustrates binding of SPIKENET to the SARS-CoV-2 protein SGP. There was highly specific binding affinity of the SPIKENET peptide to the ACE2 binding domain of SGP, shown in FIG. 2. The topography of these interactions is illustrated in variable (to illustrate various advantageous aspects) in FIGS. 3-6. FIG. 7 shows the non-bonded interactions between the binding site residues of the RBD domains and SPIKENET peptide. Consistent with this, SPIKENET is a competitive inhibitor of the spike protein (S1). This mechanistic binding affinity study by a computational approach described herein has important implications for the management of patients with SARS-CoV-2. The findings represent a new therapeutic approach for treating SARS-CoV-2 infection. Thus, the invention provides methods for treating or preventing SARS-CoV-2 infection in a human by administering to the human a therapeutically effective amount of a pharmaceutical formulation as provided by the invention. Administration can be achieved by any efficacious manner known to those with skill in the art, including but not limited to aerosol administration to lung.

Methods for synthesizing peptides are known in the art. For example, peptides can be produced using solid phase peptide synthesis on Rink amide resin with Fmoc chemistry and HBTU activation. Resulting resin-bound peptides can then be cleaved and side chains deprotected with TFA/TIPS/FhO (95:2.5:2.5) and precipitated with cold ether. Crude peptides can be purified by RP-HPLC to −98% purity). Purified peptides can be confirmed by mass spectrometry (MS) on a Bruker Esquire Ion Trap electrospray mass spectrometer, and peptide stocks lyophilized for storage at −20° C. Peptide stocks can then be thawed and reconstituted in filtered deionized distilled water to a concentration of 2 mg/mL. See, for example, Stowikowski, M. & Fields, G. B. Introduction to Peptide Synthesis. Curr. Protoc. Prot. Sci. Unit-18.1 (2002; incorporated by reference).

In an alternate method, peptides can be synthesized by standard solid phase peptide synthesis technique on Wang resin using F-moc chemistry and HBTU activation. Automated peptide synthesis, for example the CSBio 336s (CSBio, USA) automated peptide synthesizer, can be used for the synthesis. The resulting resin-bound peptides can then be cleaved and side-chain-deprotected using Reagent K (trifluoroacetic acid/thioanisole/hhO/phenol/ethanedithiol (87.5:5:5:2.5)) and, precipitated by cold ether. The crude peptides obtained are advantageously purified by reverse phase high performance liquid chromatography up to a >98% purity (Gemini 10. mu. C18 110A column). The masses of the purified peptides are checked by mass spectroscopy using a MALDI-TOF mass spectrometer. One of skill in the art will recognize additional techniques can be used to generate the peptides disclosed. SPIKENET has shown highly specific binding affinity to the ACE2 binding domain of spike glycoprotein (S1) as well as efficacy for use in both in vitro and in vivo. In addition, SPIKENET can effectively prevent SARS-CoV-2 endocytosis. SPIKENET has broader uses as a potent agent to treat any disease that display severe oxidative stress related to SPIKENET's protective effect against various toxins induced generation of free radicals. Accordingly, SPIKENET can be used to treat cancer, Alzheimer's disease, Parkinson's disease, Diabetes, cardiovascular conditions including, but not limited to hypertension, atherosclerosis, stroke, asthma, and male infertility. SPIKENET is also protective against multi-organ failure, and as such can be used for many life-threatening conditions that exhibit multi-organ failure such as sepsis, systemic inflammatory response syndrome, GI bleeding, kidney diseases and respiratory failure, graft versus host disease, radiation injury. The Neuroprotective effects of SPIKENET make it an ideal candidate to treat many neurological conditions: these include ischemic brain injury, traumatic brain injury, perinatal brain injury, various encephalopathies, Alzheimer's disease, neurodegenerative diseases, autism spectrum disease, neuromuscular disorders.

In further aspects of the disclosure are compositions and/or pharmaceutical formulation comprising a therapeutically effective amount of a peptide and one or a plurality of excipients, adjuvants, or other formulation components for treating or preventing coronavirus infection, in a subject. In certain embodiments, the formulation comprises peptides having an amino acid sequence comprising:

(M)nVR(I/L)KP(G/A)(S/T)(A/G)NKP(S/T)(D/E)D, (SEQ ID NO. 20) wherein n=0 or 1, wherein the peptide does not have the amino acid sequence MVRIKPGSANKPSDD and wherein variant amino acid sequence residues are set forth in the alternative at each substituted position. In other aspects, the pharmaceutical formulations comprise variant peptides having an amino acid sequence comprising:
MVRIKPASANKPSDD (SEQ ID NO: 1); MVRIKPGTANKPSDD (SEQ ID NO: 2); MVRIKPGTANKPTDD (SEQ ID NO: 3); MVRIKPGSGNKPSDD (SEQ ID NO: 4); MVRIKPATANKPSDD (SEQ ID NO: 5); MVRIKPGTGNKPSDD (SEQ ID NO: 6); MVRIKPGTGNKPTDD (SEQ ID NO: 7); MVRIKPATANKPTDD (SEQ ID NO: 8); MVRIKPATGNKPTDD (SEQ ID NO: 9); MVRIKPATGNKPSDD (SEQ ID NO: 10); VRLKPASANKPSED (SEQ ID NO: 11); VRLKPASGNKPSED (SEQ ID NO: 12); VRLKPGSGNKPSED (SEQ ID NO: 13); VRLKPATGNKPTED (SEQ ID NO: 14); VRLKPGTANKPTED (SEQ ID NO: 15); VRLKPATAN KPTED (SEQ ID NO: 16). VRLKPGTGNKPTED (SEQ ID NO: 17); VRLKPGTANKPSED (SEQ ID NO: 18); or VRIKPGTANKPSED (SEQ ID NO: 19) or salts or derivatives thereof. The variants described herein could be used to treat infection or sequelae thereof relating to impairment of skeletal, muscular, nervous, endocrine, cardiovascular, lymphatic, reparatory, digestive, urinary, or reproductive function.

### Methods of Treatment

Methods for using such pharmaceutical formulations are provided for treating and preventing viral disease. More specifically in one embodiment is a method of treating or preventing infection of a long term coronavirus infection comprising administering a peptide comprising the amino acid sequence set forth in SEQ ID NO: 20. In some embodiments, the methods described herein comprise administering a peptide comprising the amino acid sequence MVRIKPASANKPSDD (SEQ ID NO: 1).

The peptide can be administered via a variety of routes including, but not limited to, intranasally, intravenously, mucosally, orally, subcutaneously, or intramuscularly.

SPIKENET dosing is disease and patient specific but can be administered up to four times per day. More specifically SPIKENET can be administered once per day, twice per day, three times per day, or four times per 24-hour period. A dosing schedule that accounts for the total SPIKENET administration and frequency of administration can be established after determination of effective dose for a given condition. Effective doses of SPIKENET can range from 0.5 mg/kg-10 mg/kg body weight.

SPIKENET can be used in numerous conditions as described herein that exhibit severe oxidative stress, edema, LPS-mediated pathologies, inflammation, and/or thrombosis or coagulation.

SPIKENET administration exhibits a significant technical effect in treating edema and progressive water accumulation in tissues. The inventors have found that, unexpectedly, SPIKENET administration significantly reduces organ edema making it an ideal peptide for use in wide-ranging conditions that exhibit similar disease progression mediating water accumulation. These conditions may include, but are not limited to venous insufficiency, heart failure, renal disease, low protein levels, liver problems, deep vein thrombosis, infections, angioedema, certain medications, lymphedema, menstruation, pregnancy, neuromyelitis optica, other viral infections, and burns.

Further unexpectedly SPIKENET demonstrates a potent effect in preventing lipopolysaccharide mediated pathophysiological alterations. Accordingly, SPIKENET administration can be extended to treat various inflammatory and autoimmune diseases. These include fatty liver disease, endometriosis, Type 2 diabetes mellitus, Type 1 diabetes mellitus, Inflammatory bowel disease (IBD), asthma, rheumatoid arthritis, obesity, SLE, antiphospholipid antibody syndrome, malignancy, etc.

SPIKENET may further be helpful in preventing or treating pregnancy complications, such as pregnancy loss, implantation failure, fetal growth restriction, preeclampsia, gestational diabetes, preterm birth, abruption, preterm rupture of membranes, cholestasis of pregnancy, ovarian hyperstimulation syndrome, thrombosis associated with pregnancy.

Also, unexpectedly the inventors demonstrate that SPIKENET prevented thrombosis induced by MHV-1 virus. This supports SPIKENET as an effective peptide in addressing coagulation and conditions caused by coagulation issues. These includes, but is not limited to, hemophilia, von Willebrand disease, clotting factor deficiencies, idiopathic thrombocytopenia purpura, hypercoagulable states, deep venous thrombosis, thrombotic thrombocytopenia purpura, malignancy, disseminated intravascular coagulation etc.

Pharmaceutical compositions described herein to be used for in vivo administration typically must be sterile. This can be accomplished by use of multiple methods, as known by one of skill in the art. Pharmaceutical compositions can also be is lyophilized and sterilized or sterilized then lyophilized. Pharmaceutical compositions for storage can be stored as solutions, suspensions, gels, emulsions, solids, or as a lyophilized powder.

The effective amount of a peptide composition to be employed therapeutically varies depending on the indication, route of administration, and body size. A typical dosage can range from about 0.005 mg/kg (5 pg/kg)-10,000 mg/kg accounting for the factors noted above. More specifically the typical dose can range from about 0.01 mg/kg g to about 10,000 mg/kg; about 0.1 mg/kg to about 10,000 mg/kg; about 1.0 mg/kg to about 10,000 mg/kg; about 10 mg/kg to about 10,000 mg/kg; about 100 mg/kg to about 10,000 mg/kg; about 250 mg/kg to about 10,000 mg/kg; about 500 mg/kg to about 10,000 mg/kg; about 1000 mg/kg to about 10,000 mg/kg; about 1500 mg/kg to about 10,000 mg/kg; about 2000 mg/kg to about 10,000 mg/kg; about 2500 mg/kg to about 10,000 mg/kg; about 3000 mg/kg to about 10,000 mg/kg; about 3500 mg/kg to about 10,000 mg/kg; about 4000 mg/kg to about 10,000 mg/kg; about 4500 mg/kg to about 10,000 mg/kg; about 5000 mg/kg to about 10,000 mg/kg; about 5500 mg/kg to about 10,000 mg/kg; about 6000 mg/kg to about 10,000 mg/kg; about 6500 mg/kg to about 10,000 mg/kg; about 7000 mg/kg to about 10,000 mg/kg; about 7500 mg/kg to about 10,000 mg/kg; or about 8000 mg/kg to about 10,000 mg/kg.

In further aspects the typical dose can range from about 0.005 mg/kg to about 10,000 mg/kg; about 0.005 mg/kg to about 5000 mg/kg; about 0.005 mg/kg to about 2500 mg/kg; about 0.005 mg/kg to about 2000 mg/kg; about 0.005 mg/kg to about 1500 mg/kg; about 0.005 mg/kg to about 1000 mg/kg; about 0.005 mg/kg to about 500 mg/kg; about 0.005 mg/kg to about 250 mg/kg; about 0.005 mg/kg to about 200 mg/kg; about 0.005 mg/kg to about 150 mg/kg; about 0.005 mg/kg to about 100 mg/kg; about 0.005 mg/kg to about 50 mg/kg; about 0.005 mg/kg to about 25 mg/kg; about 0.005 mg/kg to about 24 mg/kg; about 0.005 mg/kg to about 23 mg/kg; about 0.005 mg/kg to about 22 mg/kg; about 0.005 mg/kg to about 21 mg/kg; about 0.005 mg/kg to about 20 mg/kg; about 0.005 mg/kg to about 19 mg/kg; about 0.005 mg/kg to about 18 mg/kg; about 0.005 mg/kg to about 17 mg/kg; about 0.005 mg/kg to about 16 mg/kg; about 0.005 mg/kg to about 15 mg/kg; about 0.005 mg/kg to about 14 mg/kg; about 0.005 mg/kg to about 13 mg/kg; about 0.005 mg/kg to about 12 mg/kg; about 0.005 mg/kg to about 11 mg/kg; about 0.005 mg/kg to about 10 mg/kg; about 0.005 mg/kg to about 9 mg/kg; about 0.005 mg/kg to about 8 mg/kg; about 0.005 mg/kg to about 7 mg/kg; about 0.005 mg/kg to about 6 mg/kg; or about 0.005 mg/kg to about 5 mg/kg; about 0.005 mg/kg to about 4 mg/kg; about 0.005 mg/kg to about 3 mg/kg; about 0.005 mg/kg to about 2 mg/kg; or about 0.005 mg/kg to about 1 mg/kg.

In further aspect the typical dose can range from about 0.005 mg/kg to about 10,000 mg/kg; about 0.1 mg/kg to about 5000 mg/kg; about 0.1 mg/kg to about 5000 mg/kg; about 0.1 mg/kg to about 2500 mg/kg; about 0.1 mg/kg to about 2000 mg/kg; about 0.1 mg/kg to about 1500 mg/kg; about 0.1 mg/kg to about 1000 mg/kg; about 0.1 mg/kg to about 500 mg/kg; about 0.1 mg/kg to about 250 mg/kg; about 0.1 mg/kg to about 200 mg/kg; about 0.1 mg/kg to about 150 mg/kg; about 0.1 mg/kg to about 100 mg/kg; about 0.1 mg/kg to about 50 mg/kg; about 0.1 mg/kg to about 25 mg/kg; about 0.1 mg/kg to about 20 mg/kg; or about 0.1 mg/kg to about 10 mg/kg.

Dosing frequency will depend upon the pharmacokinetic parameters of the peptide with consideration of the disease. The therapeutic composition can be administered as a single dose or in two or more doses which may contain the same or different amount of the peptide at any time during a 24-window. Dosing parameters can be established using dose-response curves as known by one of skill in the art.

The peptide and/or pharmaceutical composition can be administered using known methods and injection routes such as intravenous, mucosally, orally, intraperitoneal, intracerebral (intraparenchymal), intracerebroventricular, intramuscular, intraocular, intraarterial, intraportal, or intralesional routes; by sustained release systems. In a preferred embodiment the peptide and/or pharmaceutical composition is administered intranasally.

### EXAMPLE

### Example 1

Diverse histological alterations were discovered during our examination of MHV-1 infection within the murine integument, probing into the acute, long-COVID, and SPK treatments (SPK is not displaying any changes in the control group, which was identical to the healthy group; the figure is not shown). Currently, no treatments are available to eliminate post-acute SARS-CoV-2 complications (PASC) or long-COVID resulting from mild to moderate SARS-CoV-2 infection. Our laboratory has developed potential therapeutic targets and strategies to address long-COVID. Drawing from the molecular structure of the SARS-CoV-2 spike glycoprotein-1 (S1) and its interaction with the angiotensin-converting enzyme-2 (ACE2) receptor for cell entry [12], we designed a peptide, termed SPIKENET (SPK), comprising 15 amino acids. Employing computational programs such as computational docking and molecular dynamic studies, we focused on the spike glycoprotein, crucial for viral entry into the cell via the ACE2 receptor [12, 13]. This synthetic peptide is engineered to specifically bind to the S1 glycoprotein, impeding SARS-CoV-2 entry into the host cell. Notably, this approach has demonstrated efficacy against various SARS-CoV-2 variants (SPK is patent-protected by the University of Miami Miller School of Medicine Office of Technology Transfer-Inventors, Arumugam R. Jayakumar, and Michael J. Paidas).

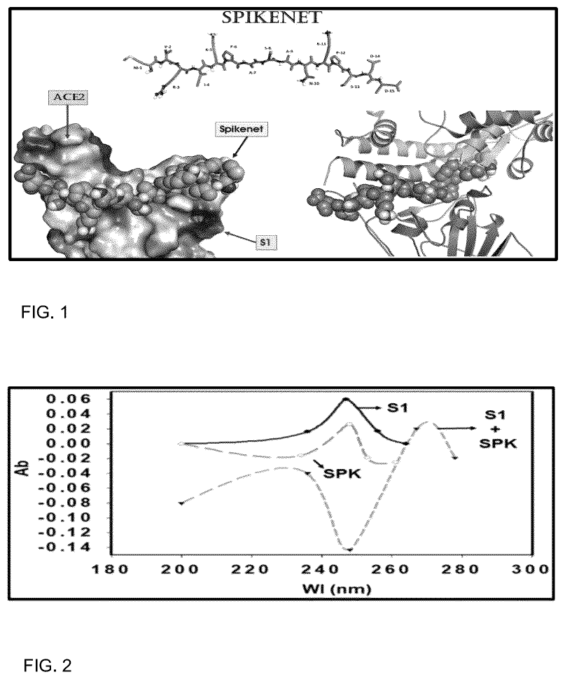

We synthesized a series of candidate peptides to target the S1 glycoprotein of the SARS-CoV-2 virus. Among these peptides, SPK exhibited the highest efficacy in binding to the ACE2 receptor. Our investigation revealed a specific binding affinity of SPK to the ACE2 binding domain of the SRAS-CoV-2 S1. The computational protein docking approach shows particular SPIKENET binding affinity to the ACE2 and CEACAM1 binding domains of the SARS-CoV-2 and MHV-1 spike glycoproteins (S1), respectively (FIG. 1) [14]. Further, our spectroscopic analysis demonstrates particular SPK binding affinity to the ACE2 binding domain of the S1 (FIG. 2). The peptide, composed of 15 amino acids, showed a theoretical mass of 1628.9 Daltons, with an observed mass of 1628.4 Daltons, indicating high purity at 98.7%. Sigma/Aldrich synthesized the peptide (Saint Louis, MO, USA).

A molecular docking investigation explored the interactions between the receptor binding domain (RBD) of SARS-CoV-2 and SPIKENET, our proprietary synthesized peptide. This study sought to improve our understanding of the non-bonded interactions, peptide conformational preferences over the human ACE2 binding site within the RBD domain of the COVID-19 virus, and the binding affinity of the peptide. Following the docking simulation, ten distinct conformations of the SPIKENET peptide were generated. Among these conformations, the model exhibiting the highest binding affinity (−156.2 kcal/mol) was selected for subsequent detailed binding analysis.

The LIGPLOT diagram illustrating the protein-peptide interaction between the RBD and SPIKENET is depicted in FIG. 3. The SPIKENET peptide residues, highlighted in pink, are presented at the top, while the amino acid residues of the RBD from SARS-CoV-2 are depicted in yellow at the bottom. Comprising 15 amino acid residues, the SPIKENET peptide engages in non-bonded interactions with 14 residues within the RBD domain of SARS-CoV-2, underscoring its significant binding affinity with the COVID-19 Spike protein [14]. Notably, our analysis of structural data reveals a striking similarity between the residues involved in SPIKENET peptide binding and those necessary for binding with human ACE2 receptors, facilitating viral infection in humans.

Further examination of the complex structure of RBD-SPIKENET interaction exhibits strong electrostatic intermolecular interactions (within the range of 2.59 to 3.06 Å distances) between specific residues of the RBD domain, including Gln-474, Lys-458, Ser-477, Tyr-473, Thr-500, Gln-498, Arg-403, Lys-417, Asp-420, and Asn-460, and residues of the SPIKENET peptide, namely Ser-13, Asp-14, Asp-15, Met-1, Arg-3, Pro-6, Ser-8, Ala-7, Asn-10, and Lys-11. Additionally, hydrophobic interactions are observed between residues Phe-456, Phe-497, Gly-476, Asn-501, Tyr-505, Gly-502, Gly-496, Tyr-449, Gln-493, Gly-447, Tyr-453, Glu-406, Leu-455, Tyr-495, and Tyr-421 and residues Pro-12, Val-2, Ile-4, and Ala-9 from the SPIKENET peptide, further enhancing peptide stability at the ACE2 binding site of the RBD [14].

For administration, the pharmaceutical formulation of SPIKENET can be delivered intravenously, transmucosal, orally, subcutaneously, or intramuscularly, with the recommended dosing frequency of twice daily for both human subjects and experimental animals. The product is supplied as a white lyophilized powder in vials containing 10 mg, devoid of preservatives, and exhibits chemical stability for 72 hours post-reconstitution when stored between −20 and −40° C. The lyophilized powder can be stored at temperatures ranging from −20 to −80° C., with an alternative storage option at −20° C. for 3-6 months and longer durations (6-48 months) at −80° C. Shelf-life information is provided on the product information sheet, and administration beyond the indicated expiration date on the package and vial is not recommended. Reconstitution of the peptide should adhere to aseptic techniques to ensure product sterility, and any unused solution should be discarded as per the Environmental Protection Agency guidelines. Based on our computational docking and spectroscopic analyses, we embarked on pilot studies to evaluate the potential protective effects of our peptide against COVID-19. Two distinct animal models of COVID-19 were selected for this investigation. The first model utilized MHV-1-induced mice to simulate COVID-19, while the second model employed humanized mice infected with SARS-CoV-2 (K18-hACE2 transgenic mouse), developed in collaboration with Dr. Bridget Barker from Northern Arizona University. The MHV-1 mouse model was previously developed in our laboratory under BSL-2 containment and documented in several publications [15-19].

After developing these animal models, pilot studies were conducted to assess the impact of SPIKENET on animal survival. These studies yielded compelling results indicative of significant protection against infection with SARS-CoV-2. Detailed data from these pilot studies are provided below.

Given that the MHV-1 virus utilizes carcinoembryonic antigen-related cell adhesion molecule-1 (CEACAM1) as its host receptor [19], our initial investigation aimed to ascertain whether our peptide exhibited binding affinity with the MHV-1 spike protein. Then, to ensure broad specificity towards various forms of spike proteins, including the different SARS-CoV-2 variants that have emerged, we engineered our peptide to target these proteins specifically. To validate this, we first confirmed the interaction of our peptide with the MHV-1 N protein's N-terminal Domain (NTD).

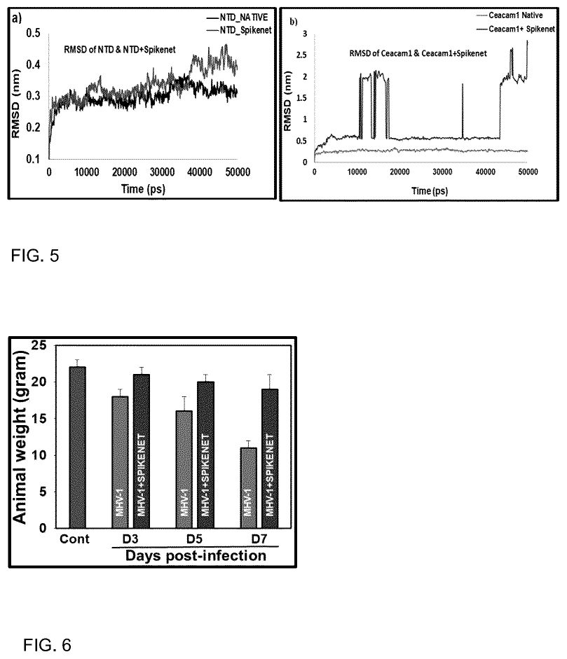

Using molecular docking and molecular dynamics simulations, we rigorously examined the binding interactions between our peptide, MHV-1 NTD, and CEACAM1. Our investigations found a robust binding affinity of SPIKENET with the CEACAM1 binding domain of the MHV-1 NTD, mirroring the affinity observed with SARS-CoV-2 S1. Alternatively, this binding was not observed with ACE2 or CEACAM1, as evidenced by both docking and molecular dynamics analyses, as illustrated in FIGS. 4 and 5. Briefly, confirmatory computer molecular dynamic studies show RMSD of both complexes with their respective native proteins (FIG. 5). The structures showed complete equilibration in the system when comparing the RMSD of both NTD and NTD+SPK (a) after 50 ns dynamic simulation. The SPK peptide was well stabilized and had a high affinity at the CCM binding location of NTD. However, the RMSD analysis of CCM and CCM+SPK structures after the 50 ns dynamic simulation exhibited more flexibility at the NTD binding site of CCM than native CCM (b), suggesting the SPK peptide detachment and displacement over the CCM.

We conducted a comprehensive assessment to determine the efficacy of our peptide in mitigating animal mortality, weight loss, clinical symptoms, and pathological alterations associated with SARS-CoV-2 infection. In addition to evaluating these significant clinical parameters and survival rates, we investigated critical pathogenic events speculated to contribute to the progression of SARS-CoV-2 infection.

These analyses encompassed the examination of body weight fluctuations (FIG. 6), multi-organ pathological changes, oxidative/nitrative stress, development of multi-organ edema, and alterations in the expression of water channel proteins aquaporins 1, 4, 5, and 8 in the MHV-1 mice model of COVID-19. Our findings suggest substantial protection against these pathological changes alongside improved animal survival (FIG. 7) when MHV-1 mice were treated with SPIKENET (5 mg/kg) three times, administered every alternate day beginning from the onset of sickness, i.e., day two post-injection [14].

The long-term sequelae of COVID-19 was investigated in these mice through 12 months post-infection. Our observations unveiled irreversible pathological alterations in multiple organs, with pronounced impairments evident in the brain, lungs, and heart compared to the liver and kidneys. Interestingly, the virus persisted in all organs tested during acute infection and in the long-term post-infection phase. Treatment of MHV-1-infected mice with SPIKENET significantly attenuated disease progression and the pathological changes observed during long-term infection. These results further emphasize COVID-19's role in provoking enduring irreversible alterations primarily affecting the brain, lungs, and heart. Additionally, our results highlight the therapeutic potential of SPIKENET in mitigating both acute and long term pathological consequences associated with SARS-CoV-2 infections (FIG. 8).

Corroborating our findings in the MHV-1 mouse model, we recently documented SARS-CoV-2 in human brain samples obtained from a prematurely born neonate delivered at our facility to a mother infected with SARS-CoV-2 [20]. This notable case involved a pregnant individual presenting to our facility at 27 weeks gestation with severe respiratory symptoms and pneumonia. Upon initial examination, she tested positive for SARS-CoV-2 via RT-PCR. The mother was then admitted to the intensive care unit (ICU) and received treatment for pneumonia and multisystem disease while undergoing continuous fetal monitoring.

Due to the deteriorating maternal clinical condition, an emergent Cesarean section was performed at 32 weeks of gestation. Immediately following delivery, the newborn exhibited seizure-like activity and respiratory distress. At 24 hours of life, COVID-19 IgG and combined IgG/IgM/IgA reactivity to a recombinant derivative of the SARS-CoV-2 spike protein were detected in the infant's serum, accompanied by markedly elevated serum inflammatory markers and cytokine levels. MRI conducted on day 2 of life revealed a left germinal matrix and left ventricular hemorrhage. Follow-up imaging at 10 weeks of life indicated resolution of bleeds, yet severe parenchymal atrophy was observed. The infant was discharged home after three months with a diagnosis of seizure disorder and acquired growth failure, with abnormal neurological examination persisting at the 12-month follow-up evaluation [20].

Tragically, at 13 months of age, the infant experienced asystolic cardiac arrest and was transported to the emergency department, where resuscitation attempts were unsuccessful. Autopsy findings revealed a significant reduction in brain weight, enlarged ventricles, reactive gliosis, and neuronal death throughout the cerebral, cerebellar, and brainstem white matter [20].

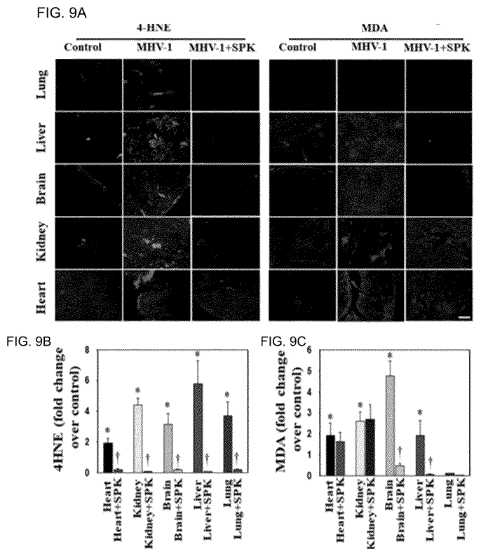

We detected the presence of both spike protein (S1) in the brain and the nucleocapsid protein, which indicates viral presence after SARS-CoV-2 infection [20]. Furthermore, we observed blood-brain barrier breakdown and reduced levels of ACE2 in the infant's brain. The discovery of SPIKENET's protective effect against the development of acute edema in MHV-1-infected mice, coupled with insights from SARS-CoV-2 infection in humans, underscores a possible link between generalized edema and the progression of COVID-19 disease. We also noted increased edema with varying severity in multiple organs, including the lung, skin, liver, brain, kidney, and heart [14, 20-22]. Treatment of MHV-1-inoculated mice with SPIKENET yielded edema levels comparable to controls on day 7 post-infection (FIG. 8). Unlike SPIKENET, the administration of another small molecular peptide (VRIKPGTANKPSED) to MHV-1-inoculated mice did not impact animal survival, consistent with its lack of binding affinity with S1 or ACE2/CEACAM-1 (Figure not shown). Taken together, these findings suggest that the development of edema in various organs may represent a seminal event in SARS-CoV-2 infection, and our newly developed peptide, SPIKENET, which effectively prevents S1 binding with ACE2 or CEACAM-1, holdS promise as a therapeutic agent targeting SARS-CoV2 infection and decreasing risk of long-term adverse effects on overall health and wellbeing (FIG. 9).

In addition to its role in preventing generalized edema, SPIKENET's efficacy against acute oxidative stress is equally noteworthy. Administration of 5 mg/kg of SPIKENET to MHV-1 infected mice reversed physiologic changes attributed to oxidative stress. While redox system alterations have been implicated in the pathophysiology of SARS-CoV-2 infection [23], our current understanding of the impact of oxidative stress and its ramifications on pathophysiological changes during infection remains limited.

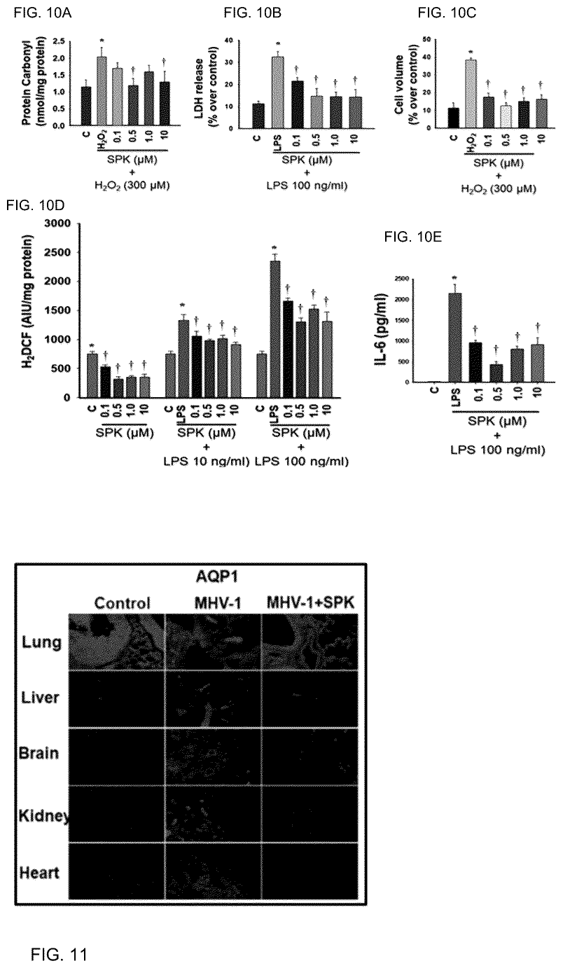

Supporting the role of oxidative stress (OS) in the development of COVID-19 disease, we identified lipid peroxidation-derived aldehydes, namely 4-hydroxynonenal (4-HNE) and malondialdehyde (MDA), in various organs of MHV-1 inoculated mice (6 days post-MHV-1) (FIG. 10A). Treatment of MHV-1-infected mice with SPIKENET (5 mg/kg), however, diminished this effect (FIG. 10B and FIG. 10C). This result offers additional evidence for the potential role of OS in the progression of SARS-CoV-2 infection.

Another hallmark of SARS-CoV-2 infection is systemic inflammation, typically associated with generating reactive oxygen species leading to OS, which is also known to contribute to disease progression [23]. Accordingly, we also investigated SPIKENET's impact on OS and inflammation. Our results indicate that SPIKENET attenuated lipopolysaccharide (LPS)-induced inflammatory response and OS in primary cultures of rat brain microglia (FIG. 11). Additionally, SPIKENET inhibited LPS-induced cell death (LDH release) and cellular stress in primary cultures of rat brain microglia, as well as chemically induced cell swelling in astrocyte cultures, a significant event observed in lungs post-SARS-CoV-2 infection (FIG. 11).

Finally, our study also assured the safety profile of SPIKENET. Exposure to SPIKENET at high concentrations (50 and 100 μm) did not compromise cell survival or mitochondrial function in brain microglia, astrocytes, and neurons, as evaluated through the MTT assay (3-(4,5-dimethylthiazol-2-yl)-2,5-diphenyl tetrazolium bromide) (data not shown) while preserving its antioxidant and anti-inflammatory properties. In summary, these findings further highlight the potential utility of SPIKENET in preventing SARS-CoV-2 infection and its associated long-term sequelae.

### Protective Effect of SPIKENET in Acute Water Channel Protein

Aquaporins (AQPs) are integral to maintaining intra and extracellular fluid balance. Overexpression of aquaporins in the plasma membrane is implicated in developing edema [24-27]. We identified increases in AQPs 1, 4, 5, and 8 in every organ tissue sample analyzed in our study. Notably, AQP1 was differentially regulated (FIG. 5). Treatment with SPIKENET (5 mg/kg) resulted in a reduction of AQPs 4, 5, and 8 levels while returning decreased AQP1 levels to normal ranges (FIG. 11) [14]. These results strongly support the central role that dysregulated AQP expression, likely secondary to oxidative stress, plays in the pathogenesis of SARS-CoV-2 infection and resultant generalized edema.

### Protective Effect of SPK on Organs During Chronic Infection

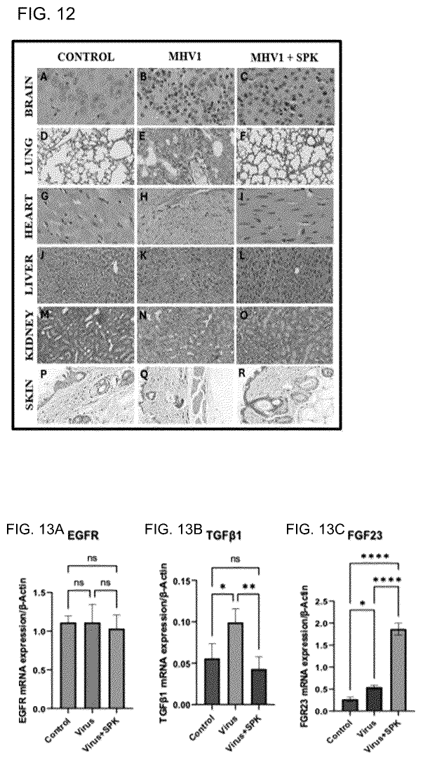

In the lungs of MHV-1-infected mice observed 12 months post-infection, we found severe lung inflammation, bronchiolar epithelial cell degradation, leukocyte infiltration, and hemosiderin-laden macrophages (FIG. 12). Additionally, interstitial edema, thickened bronchiolar airways due to fibrotic remodeling, increased numbers of goblet cells in the epithelial lining of bronchioles, and enhanced inflammatory cell infiltration in bronchiole walls were noted [8]. Treatment with SPIKENET reversed some of these findings (FIG. 6).

In the hearts of long-term COVID models, we observed severe interstitial edema, vascular congestion and dilation, and infiltration of red blood cells (RBCs) between degenerative myocardial fibers (FIG. 12). Additionally, there were inflammatory cell infiltrates, apoptotic hypertrophy, and fibrosis [8]. These changes were seen during both acute and long-term conditions. SPIKENET treatment, however, effectively blunted these changes at both time-points (FIG. 12).

In the livers of extended COVID models of MHV-infected mice, we detected severe vascular congestion, hepatocyte degeneration, luminal thrombosis of the portal and sinusoidal vessels, hemorrhagic changes, and cell necrosis (FIG. 12). Furthermore, there was increased lymphocyte infiltration in sinusoidal spaces, multifocal hepatic necrosis in periportal areas and near terminal hepatic veins, an increased number of portal veins associated with luminal severe dilation, activated Kupfer cells containing necrotic debris, eosinophilic bodies, mitotic cells, balloon-like liver cells, mild lobular lymphocytic inflammation, mild portal tract inflammation, and mild hydropic degeneration of liver parenchymal cells. Additionally, liver enzyme concentrations (ALT, ALP, AST, and Bilirubin) were significantly elevated compared to the control [8]. Again, treatment with SPIKENET diminished the extent of these pathological changes and facilitated the restoration of liver enzyme levels to normal ranges (FIG. 12) [8].

### Protective Effect of SPK in Renal Fibrosis

Growth factors and their associated cellular signaling pathways have been implicated in the pathogenesis and progression of fibrosis in various organs [28-31]. Pulmonary fibrosis is seen in individuals infected with SARS-CoV 2 and is believed to be mediated by the epidermal growth factor receptor (EGFR) signaling pathway [28, 29]. Our earlier histopathological analyses of the kidney illustrated severe and irreversible kidney dysfunction in both short and long-term post-infection periods [21]. While EGFR stimulation is associated with renal fibrosis in other clinical conditions [30], its role during acute and long-term SARS-CoV-2 infection is not fully understood. We employed our surrogate mouse model to determine the extent to which EGFR stimulation is responsible for renal fibrosis in COVID-19 disease. We determined that EGFR levels exhibited no significant alterations in long COVID models regardless of treatment with SPIKENET (FIG. 13) [21].

Transforming growth factor-beta (TGF-β) is widely recognized as a critical contributor to fibrosis development and progression in various organs. Its involvement primarily entails activation of canonical and non-canonical signaling pathways, resulting in deposition of extracellular matrix (ECM) [30, 31]. We found a significant increase in TGF-β mRNA levels in kidneys 12 months post-MHV-1 infection (FIG. 13) [21], supporting its involvement in developing renal fibrosis during cases of long COVID. On the other hand, TGF-β mRNA levels in MHV-1 infected mice treated with SPIKENET were significantly reduced (FIG. 13), offering additional evidence for the integral role TGF-β plays in renal fibrosis secondary to SARS-CoV-2 infection [21].

Another growth factor, FGF23, is known to rise in response to inflammation and hypoxia and can contribute to the development of chronic kidney disease (CKD) [32]. Notably, elevated FGF23 levels have been documented in COVID-19-infected patients with a history of CKD [33]. In the long-term post-infection group, we observed a significant increase in FGF23 expression, further exacerbated by treatment with SPK (5 mg/kg) (FIG. 13) [21]. While the reason for such an effect of SPK on FGF23 is unclear, we speculate that SPK may have increased the FGF23 by mechanisms independent of inflammatory stimulation.

### Protective Effect of SPK in Skin

Remarkably, treating MHV-1-infected mice with the synthetic peptide SPK substantially moderated cutaneous changes associated with long COVID [22]. SPK intervention effectively restored hair follicle morphogenesis (FIG. 12) in stark contrast with the untreated control group (FIG. 12). Intriguingly, this therapeutic intervention reinstated architectural integrity in adipose tissue and across dermal and epidermal layers, including sebaceous glands (FIG. 12), compared to the control group (FIG. 12). Additionally, sebaceous glands exhibited notable dispersion along hair follicle lengths, accompanied by discernible thickening of the panniculus carnosus (FIG. 6), notably absent in untreated controls (FIG. 12). In FIG. 11Q, diverse stages of hair follicle degeneration are evident long-term post-infection, juxtaposed with standard, healthy mice, as illustrated in FIG. 12. The application of SPK treatment led to the restoration of hair follicles across distinct stages, coupled with adipose tissue regeneration. SPK treatment also reversed cutaneous abnormalities observed in chronic infection, restored hair follicle numbers, and reorganized the architecture of epidermal and dermal layers, repairing adipose tissue [22]. As observed previously in the kidney, SPK administration reduced TGF-β levels. TGF-β initiates an intercellular signaling cascade that stimulates fibroblasts responsible for collagen production deposited in the ECM. This suggests SPK can mitigate collagen and fibrosis accumulation in the ECM by regulating TGF-β [22].

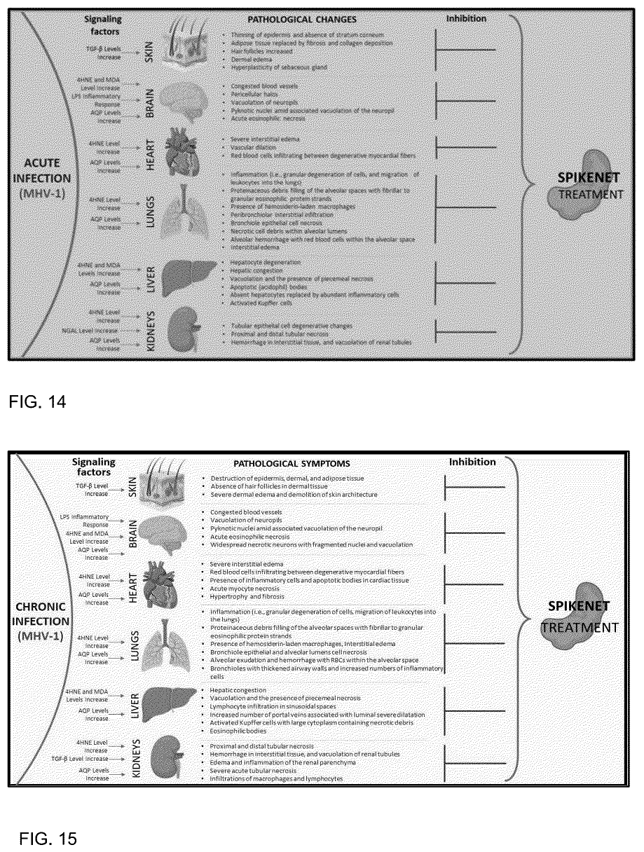

Our investigations increasingly demonstrate that administering SPK effectively prevents both acute and long-term complications of COVID-19 (FIGS. 14 & 15). These findings suggest SPK addresses immediate symptoms and complications associated with acute COVID-19 infection and is crucial in mitigating the risk of long-term health issues post-infection. This discovery holds significant promise for improving public health outcomes and reducing the burden on healthcare systems worldwide. Furthermore, the efficacy of SPK in preventing COVID-19 complications underscores the importance of continued research and development efforts in identifying effective therapeutic interventions against emerging infectious diseases.

In summary, SPIKENET (SPK) shows significant potential as a treatment for diminishing and alleviating symptoms of acute and long COVID-19 in humans, as demonstrated in the MHV1 mice model. The mechanism of SPK binding to SARS-CoV-2 spike glycoprotein-1 (S1), preventing S1 from binding to the human ACE2 receptor, is implied to inhibit viral entry into host cells. Additionally, SPK mitigated abnormal protein expression in many organs, attenuating their acute and long-term COVID symptoms, such as edema, renal fibrosis, and tissue necrosis. However, further studies are warranted to observe further the effects of SPIKENET on other vital organs and its efficacy on other COVID models to underscore its applicability as a chronic COVID treatment.

### Example 2

Congenital malformation in murine hepatitis virus-1 beta coronavirus mouse model is induced by the severity of viral load and is mediated by placental damage: Protective effect of SPK.

Children born to mothers infected with COVID-19 during pregnancy may face an increased risk of neurological complications. This risk arises from the structural and functional brain alterations revealed by MRI, PET, and SPECT imaging following SARS-CoV-2 infection. The nature of SARS-CoV-2 infection during pregnancy, particularly its impact on neonates, has been a topic of significant uncertainty. This ambiguity underscores the importance of our recent reports, which provide crucial insights. Studies have demonstrated definite vertical transmission and placental abnormalities following maternal SARS-CoV-2 infection. We now show that acute murine hepatitis virus-1 (a beta coronavirus) infection in mice results in major congenital malformations. These include central nervous system malformations (brain/face, craniothoracopagus twinning, and neural tube defects) and abdominal wall defects (gastroschisis/omphalocele). Central nervous system defects predominate in these pups compared to other potential abnormalities. While these changes occur with viral load, no malformations were identified in pups born to these mice when treated with SPK (1 mg/kg), as demonstrated earlier by us.

Next-generation sequencing of the placenta from virus-infected mice shows differential gene expression known to affect neonatal development. These include Mchr1, mdk, IGTP, NR4A1, STAT3, JPT2, Krt18, IRF7, Dlk1, USP2/18, Lfit1, ZBP1, XAF1, AKAP12, CAV1, LGALS3BP, and Socs3/JAK1/STAT3 pathway, along with FOXF1, S100A8/A9, Nrp3, EDN1, Zfp36L1, CHIKV, HCMV, HCV, DENV, WNV, SINV, and Rsad2, which are known to affect the placenta under various conditions, including preeclampsia, hypoxia, infection, and inflammation. Some of the newly upregulated genes in high virus-inoculated mice are Irmg1, Gbp2, Gbp2b, ligp1, predicted gene Gm20412, phosphomevalonate kinase, Klf2, Ifis44l, Tor3a, and Oas1a (involved in the innate immune response and inflammation to viral infection), Oasl2 (which differentially regulates RNA and DNA virus replication), Olfr56 (adenosine receptor ligands), Dtx31, STAT1/STAT2, Tap1, Lrrc32, Ifi47, CAAA01077340, Hist1h2br, Parp9/12, Rnf225, Exoc312, Gbp3, Rtp4, Uba7, Errfi1, Snrk, Hist1h2bq, and Parp10. Many downregulated genes, particularly the transporters, are also identified in high virus-inoculated mice placentas and are known to affect fetal development. These include Slc13a4, Slc7a2, Slc41a1, and Slc45a3, Folr1, as well as Kcnk16, Dennd1b, Dipk1a, Usp31, Zfp385c, Trank1, Pigg, Snx7, Zdhhc2, Dennd4a, Ccdc43, Epn2, Tesk2, Itpka, Lars2, Pdzd3, Bmp10, and Gm27179. Notably, treating pregnant mice with SPK didn't affect (i.e., up or downregulate) these genes (selected genes as measured by RT-PCR).

Studies have shown that the X chromosome plays an essential role in the development of psychiatric disorders (Goldstein et al., 2013; Perrin et al., 2010; Crow, 2013; Ross et al., 2001; Ross et al., 2006). The Xist gene (X-inactive specific transcript) is involved in X chromosome inactivation. Overexpression of XIST affects X-chromosome inactivation and leads to the upregulation of X-linked escape genes responsible for the development of psychiatric disorders, especially major affective disorders such as bipolar disorder and depression (Ji et al., Ebio Medicine, 2:909-918, 2015). We found this gene overexpressed in the brains of pups born to MHV-1-infected mice. It is well known that several neurological disorders can be sex-biased and have a higher prevalence in men than in women. Data suggest that Xist gene upregulation in males might be linked to Alzheimer's disease and Parkinson's disease in men (Jianjian Li, Genes Dis., 9:1478-1492, 2022). Treating pregnant mice with SPK didn't affect these genes in these pups.

### Mice Body Weight Change and Effect of SPK

Mice given MHV-1 (5000 PFU) showed weight gain compared to untreated mice (see below). Such weight gain was not observed in mice treated with SPK.

| Females | | |
|---|---|---|
| Bw 9w | 19.6 | g |
| No MHV-1 | 27.6 | g |
| MHV-1 | 34.1 | g |
| +SPK | 29.7 | |
| Males | | |
| Bw 9w | 22.4 | g |
| No MHV-1 | 33.8 | g |

| | |
|---|---|
| MHV-1 | 38.2 |
| +SPK | 34.1 |

### Therapeutic Effect of SPIKENET

Accumulating evidence indicates that SARS-CoV-2 infection results in long-term multi-organ complications, with the kidney being a primary target. This study aimed to characterize the long-term transcriptomic changes in the kidney following coronavirus infection using a murine model of MHV-1-induced SARS-like illness and to evaluate the therapeutic efficacy of SPIKENET.

Methods: A/J mice were infected with MHV-1. Renal tissues were collected and subjected to Next Generation RNA Sequencing to identify differentially expressed genes associated with chronic infection. Bioinformatic analyses, including PCA, volcano plots, and GO/KEGG pathway enrichment, were performed. A separate cohort received SPIKENET treatment, and comparative transcriptomic profiling was conducted.

Results: Long-term MHV-1 infection resulted in sustained upregulation of genes involved in muscle regeneration, cytoskeletal remodeling, and fibrotic responses. Notably, both expression and variability of SLC22A6 and 3SLC22A8, key proximal tubule transporters, were reduced, suggesting a loss of segment-specific identity. SLC transporters also exhibited expression patterns consistent with tubular dysfunction and inflammation. These findings suggest aberrant activation of myogenic pathways and structural proteins in renal tissues, consistent with a pro-fibrotic phenotype. In contrast, SPIKENET treatment reversed the expression of most genes, restoring gene profiles toward those observed in control mice.

Conclusion: MHV-1-induced long COVID is associated with persistent transcriptional reprogramming in the kidney, indicative of chronic inflammation, cytoskeletal dysregulation, and fibrogenesis. SPIKENET demonstrates robust therapeutic potential by normalizing these molecular signatures and preventing long-term renal damage. These findings underscore the relevance of the MHV-1 model and support SPIKENET as a therapy for COVID-19-associated renal sequelae.

### REFERENCES

1. World Health Organization. Who Coronavirus (COVID-19) Dashboard. World Health Organization. Available online: https://covid19.who.int/(accessed on 10 Feb. 2024).
2. Centers for Disease Control and Prevention. (n.d.). SARS-CoV-2 variant classifications and definitions. Centers for Disease Control and Prevention. https://www.cdc.gov/coronavirus/2019-ncov/variants/variant-classifications.html
3. Davis, H. E.; McCorkell, L.; Vogel, J. M.; Topol, E. J. Long COVID: major findings, mechanisms and recommendations. Nat Rev Microbiol 2023, 21, 133-146. doi: 10.1038/s41579-022-00846-2. Epub 2023 Jan. 13. Erratum in: Nat Rev Microbiol 2023, 21, 408.
4. Caldera-Crespo, L. A.; Paidas, M. J.; Roy, S.; Schulman, C. I.; Kenyon, N. S.; Daunert, S.; Jayakumar, A. R. Experimental Models of COVID-19. Front Cell Infect Microbiol 2022, 11:792584. doi: 10.3389/fcimb.2021.792584.
5. Baj, J.; Karakuła-Juchnowicz, H.; Teresiński, G.; Buszewicz, G.; Ciesielka, M.; Sitarz, R.; Forma, A.; Karakuła, K.; Flieger, W.; Portincasa, P.; Maciejewski, R. COVID-19: Specific and Non-Specific Clinical Manifestations and Symptoms: The Current State of Knowledge. J Clin Med 2020, 9, 1753. doi: 10.3390/jcm9061753.
6. Rodebaugh, T. L.; Frumkin, M. R.; Reiersen, A. M.; Lenze, E. J.; Avidan, M. S.; Miller, J. P.; Piccirillo, J. F.; Zorumski, C. F.; Mattar, C. Acute Symptoms of Mild to Moderate COVID-19 Are Highly Heterogeneous Across Individuals and Over Time, Open Forum Infectious Diseases 2021, 8, ofab090, https://doi.org/10.1093/ofid/ofab090
7. Guan, W. J.; Ni, Z. Y.; Hu, Y.; Liang, W. H.; Ou, C. Q.; He, J. X.; Liu, L.; Shan, H.; Lei, C. L.; Hui, D. S. C.; Du, B.; Li, L. J.; Zeng, G.; Yuen, K. Y.; Chen, R. C.; Tang, C. L.; Wang, T.; Chen, P. Y.; Xiang, J.; Li, S. Y.; Wang, J. L.; Liang, Z. J.; Peng, Y. X.; Wei, L.; Liu, Y.; Hu, Y. H.; Peng, P.; Wang, J. M.; Liu, J. Y.; Chen, Z.; Li, G.; Zheng, Z. J.; Qiu, S. Q.; Luo, J.; Ye, C. J.; Zhu, S. Y.; Zhong, N. S. China Medical Treatment Expert Group for Covid-19. Clinical Characteristics of Coronavirus Disease 2019 in China. N Engl J Med 2020, 382, 1708-1720. doi: 10.1056/NEJMoa2002032.
8. Paidas, M. J.; Cosio, D. S.; Ali, S.; Kenyon, N. S.; Jayakumar, A. R. Long-Term Sequelae of COVID-19 in Experimental Mice. Mol Neurobiol 2022, 59, 5970-5986. doi: 10.1007/s12035-022-02932-1.
9. Centers for Disease Control and Prevention. (n.d.-a). Covid-19 treatments and medications. Centers for Disease Control and Prevention. https://www.cdc.gov/coronavirus/2019-ncov/your-health/treatments-for-severe-illness.html
10. Davis, H. E.; McCorkell, L.; Vogel, J. M.; Topol, E. J. Long COVID: major findings, mechanisms and recommendations. Nat Rev Microbiol 2023, 21, 133-146. doi: 10.1038/s41579-022-00846-2. Epub 2023 Jan. 13. Erratum in: Nat Rev Microbiol 2023, 21, 408.
11. Centers for Disease Control and Prevention. (n.d.-b). Long Covid or post-covid conditions. Centers for Disease Control and Prevention. https://www.cdc.gov/coronavirus/2019-ncov/long-term-effects/index.html
12. Scialo, F.; Daniele, A.; Amato, F.; Pastore, L.; Matera, M. G.; Cazzola, M.; Castaldo, G.; Bianco, A. ACE2: The Major Cell Entry Receptor for SARS-CoV-2. Lung. 2020, 198, 867-877. doi: 10.1007/s00408-020-00408-4.
13. Hoffmann, M.; Kleine-Weber, H.; Schroeder, S.; Krüger, N.; Herrler, T.; Erichsen, S.; Schiergens, T. S.; Herrler, G.; Wu, N. H.; Nitsche, A.; Müller, M. A.; Drosten, C.; Pohlmann, S. SARS-CoV-2 Cell Entry Depends on ACE2 and TMPRSS2 and Is Blocked by a Clinically Proven Protease Inhibitor. Cell 2020, 181, 271-280.e8. doi: 10.1016/j.cell.2020.02.052.
14. Paidas, M. J.; Sampath, N.; Schindler, E. A.; Cosio, D. S.; Ndubizu, C. O.; Shamaladevi, N.; Kwal, J.; Rodriguez, S.; Ahmad, A.; Kenyon, N. S.; Jayakumar, A. R. Mechanism of Multi-Organ Injury in Experimental COVID-19 and Its Inhibition by a Small Molecule Peptide. Front Pharmacol 2022, 13, 864798. doi: 10.3389/fphar.2022.864798.
15. Masciarella, A. D.; Di Gregorio, D. M.; Ramamoorthy, R.; Hussain, H.; Jayakumar, A. R.; Paidas, M. J. A Mouse Model of MHV-1 Virus Infection for Study of Acute and Long COVID Infection. Curr Protoc 2023, 3, e896. doi: 10.1002/cpz1.896.
16. Oliveira, G. P.; Kroon, E. G. Mouse hepatitis virus: A betacoronavirus model to study the virucidal activity of air disinfection equipment on surface contamination. J Virol Methods 2021, 297, 114274. doi: 10.1016/j.jviromet.2021.114274.
17. Khanolkar, A.; Pewe, L.; Tifrea, D.; Perlman, S.; Harty, J. T. Developing MHV-1 infection as an animal model for severe acute respiratory syndrome (SARS) (43.30). J Immunol 1 2007, 178 (1_Supplement), S42. https://doi.org/10.4049/jimmunol. 178.Supp.43.30
18. De Albuquerque, N.; Baig, E.; Ma, X.; Zhang, J.; He, W.; Rowe, A.; Habal, M.; Liu, M.; Shalev, I.; Downey, G. P.; Gorczynski, R.; Butany, J.; Leibowitz, J.; Weiss, S. R.; McGilvray, I. D.; Phillips, M. J.; Fish, E. N.; Levy, G. A. Murine hepatitis virus strain 1 produces a clinically relevant model of severe acute respiratory syndrome in A/J mice. J Virol 2006, 80 (21): 10382-94. doi: 10.1128/JVI.00747-06.
19. Phillips, J. M.; Weiss, S. R. Pathogenesis of neurotropic murine coronavirus is multifactorial. Trends Pharmacol Sci 2011, 32, 2-7. doi: 10.1016/j.tips.2010.11.001.
20. Benny, M.; Bandstra, E. S.; Saad, A. G.; Lopez-Alberola, R.; Saigal, G.; Paidas, M. J.; Jayakumar, A. R.; Duara, S. Maternal SARS-CoV-2, Placental Changes and Brain Injury in 2 Neonates. Pediatrics 2023, 151, e2022058271. doi: 10.1542/peds.2022-058271.
21. Ramamoorthy, R.; Hussain, H.; Ravelo, N.; Sriramajayam, K.; Di Gregorio, D. M.; Paulrasu, K.; Chen, P.; Young, K.; Masciarella, A. D.; Jayakumar, A. R.; Paidas, M. J. Kidney Damage in Long COVID: Studies in Experimental Mice. Biology (Basel) 2023, 12, 1070. doi: 10.3390/biologyl2081070.
22. Hussain, H.; Paidas, M. J.; Ramamoorthy, R.; Fadel. A.; Ali, M.; Chen. P.; Jayakumar, A. R. 2024. Dermatologic Changes in Experimental Model of Long COVID. Microorganisms 2024, 2, 272. https://doi.org/10.3390/microorganisms12020272
23. Forcados, G. E.; Muhammad, A.; Oladipo, O. O.; Makama, S.; Meseko, C. A. Metabolic Implications of Oxidative Stress and Inflammatory Process in SARS-CoV-2 Pathogenesis: Therapeutic Potential of Natural Antioxidants. Front Cell Infect Microbiol 2021, 11, 654813. doi: 10.3389/fcimb.2021.654813
24. Johansson, I.; Karlsson, M.; Shukla, V. K.; Chrispeels, M. J.; Larsson, C.; Kjellbom, P. Water transport activity of the plasma membrane aquaporin PM28A is regulated by phosphorylation. Plant Cell 1998, 10, 451-9. doi: 10.1105/tpc. 10.3.451.
25. Ma, T.; Yang, B.; Gillespie, A.; Carlson, E. J.; Epstein, C. J.; Verkman, A. S. Generation and phenotype of a transgenic knockout mouse lacking the mercurial-insensitive water channel aquaporin-4. J Clin Invest 1997, 100, 957-62. doi: 10.1172/JCI231.
26. Rama Rao, K. V.; Jayakumar, A. R.; Tong, X.; Curtis, K. M., Norenberg, M. D. Brain aquaporin-4 in experimental acute liver failure. J Neuropathol Exp Neurol 2010, 69, 869-79. doi: 10.1097/NEN.0b013e3181ebe581.
27. Wittekindt, O. H., Dietl, P. Aquaporins in the lung. Pflugers Arch 2019, 4, 519-532. doi: 10.1007/s00424-018-2232-y.
28. Venkataraman, T.; Frieman, M. B. The role of epidermal growth factor receptor (EGFR) signaling in SARS coronavirus-induced pulmonary fibrosis. Antivir. Res. 2017, 143, 142-150. doi: 10.1016/j.antiviral.2017.03.022
29. Schramm, F.; Schaefer, L.; Wygrecka, M. EGFR Signaling in Lung Fibrosis. Cells 2022, 11, 986. doi: 10.3390/cells11060986.
30. Chen, J.; Chen, J. K.; Nagai, K.; Plieth, D.; Tan, M.; Lee, T. C.; Threadgill, D. W.; Neilson, E. G.; Harris, R. C. EGFR signaling promotes TGFβ-dependent renal fibrosis. J. Am. Soc. Nephrol 2012, 23, 215-224. doi: 10.1681/ASN.2011070645.
31. Sisto, M.; Ribatti, D.; Lisi, S. Organ Fibrosis and Autoimmunity; The Role of Inflammation in TGFß-Dependent EMT. Biomolecules 2021, 11, 310. doi: 10.3390/biom11020310.
32. Francis, C.; David, V.; Inflammation regulates fibroblast growth factor 23 production. Curr. Opin. Nephrol. Hypertens 2016, 25, 325-332. doi: 10.1097/MNH.0000000000000232.
33. Toro, L.; Michea, L.; Parra-Lucares, A.; Mendez-Valdes, G.; Villa, E.; Bravo, I.; Pumarino, C.; Ayala, P.; Sanhueza, M. E.; Torres, R.; et al. High Plasma Levels of Fibroblast Growth Factor 23 Are Associated with Increased Risk of COVID-19 in End-Stage Renal Disease Patients on Hemodialysis: Results of a Prospective Cohort. Toxins 2023, 15, 97. doi: 10.3390/toxins 15020097
34. Argyropoulos, C. P.; Chen, S. S.; Ng, Y. H.; Roumelioti, M. E.; Shaffi, K.; Singh, P. P.; Tzamaloukas, A. H. Rediscovering Beta-2 Microglobulin as a Biomarker across the Spectrum of Kidney Diseases. Front. Med 2017, 4, 73. doi: 10.3389/fmed.2017.00073.
35. Dimitrov, D. S. Therapeutic proteins. Methods Mol Biol 2012, 899, 1-26. doi: 10.1007/978-1-61779-921-1_1.
36. Marković, V.; Szczepańska, A.; Berlicki, Ł.; Antiviral Protein-Protein Interaction Inhibitors. J Med Chem 2024, 67, 3205-3231. doi: 10.1021/acs.jmedchem.3c01543.
37. Pozzi, C.; Vanet, A.; Francesconi, V.; Tagliazucchi, L.; Tassone, G.; Venturelli, A.; Spyrakis, F.; Mazzorana, M.; Costi, M. P.; Tonelli, M. Antitarget, Anti-SARS-CoV-2 Leads, Drugs, and the Drug Discovery-Genetics Alliance Perspective. J Med Chem 2023; 66, 3664-3702. doi: 10.1021/acs.jmedchem.2c01229.
38. Shah, D. K. Pharmacokinetic and pharmacodynamic considerations for the next generation protein therapeutics. J Pharmacokinet Pharmacodyn 2015, 42, 553-71. doi: 10.1007/s10928-015-9447-8.
39. Ebrahimi, S. B.; Samanta, D. Engineering protein-based therapeutics through structural and chemical design. Nat Commun 2023, 14, 2411. https://doi.org/10.1038/s41467-023-38039-x
40. Glassman, P. M.; Balthasar, J. P. Physiologically-based modeling of monoclonal antibody pharmacokinetics in drug discovery and development. Drug Metab Pharmacokinet 2019, 34, 3-13. doi: 10.1016/j.dmpk.2018.11.002.

What is claimed is:

1. A method of treating or preventing a long term coronavirus infection in a subject in need thereof comprising a therapeutically effective amount of a peptide, wherein the formulation comprises peptides having an amino acid sequence comprising:
(M)nVR(I/L)KP(G/A)(S/T)(A/G)NKP(S/T)(D/E)D, (SEQ ID NO: 20), where n=0 or 1.

2. The method of claim 1, wherein the peptide comprises the amino acid sequence MVRIKPASANKPSDD (SEQ ID NO: 1).
